(19) United States
(12) Patent Application Publication
(10) Pub. No.: US 2026/0233228 A1
(43) Pub. Date: Aug. 13, 2026

(54) SAMPLE VIAL CONSUMABLE FOR CYTOPATHOLOGY

(71) Applicants: ASP Health Inc., Evanston, IL (US); Planet Innovation, Box Hill VIC (AU)

(72) Inventors: Hariharan SUBRAMANIAN, Evanston, IL (US); Alex BLUESTONE, Evanston, IL (US); Tamara SWANSON, Evanston, IL (US); Blake WALLACE, Evanston, IL (US); John HART, Evanston, IL (US); Margaux HAYES, Box Hill VIC (AU); Matthew TURNER, Box Hill VIC (AU); Chester HENDERSON, Box Hill VIC (AU); Michael HENNING, Box Hill VIC (AU); Kate COSMAN, Box Hill VIC (AU); Sohail ABDULLA, Box Hill VIC (AU); Nick MOUSKOS, Box Hill VIC (AU); Santiago Beltran DIAZ, Box Hill VIC (AU)

(21) Appl. No.: 19/471,265

(22) PCT Filed: Mar. 29, 2024
(86) PCT No.: PCT/US2024/022227

§ 371 Date: Sep. 30, 2025

Related U.S. Application Data
(60) Provisional application No. 63/493,581, filed on Mar. 31, 2023.

(51) Int. Cl. B01L 3/00; A61B 10/00; B01L 9/00; G01N 1/28

(52) U.S. Cl. CPC B01L 3/505; A61B 10/0045; B01L 9/00; G01N 1/286; B01L 2200/087; B01L 2200/12; B01L 2300/021; B01L 2300/043; B01L 2300/123; G01N 2001/2866

(57) ABSTRACT

Embodiments of the disclosed technology are directed to consumable components for cytology, which include a sample vial. In an example, the consumable sample vial is designed to receive a sample volume between 10 and 750 μL, receive a buffer solution, and enable mixing of the sample with the buffer solution prior to allowing a user to deposit some amount of the mixture into another consumable for subsequent processing, e.g., cell deposition and staining. The remaining mixture may be securely stored and transported using the sample vial. The sample vial is manufactured from a transparent and flexible material that allows the user to observe the fluid and mixture levels in the vial and squeeze to dispense the mixture.

### CROSS-REFERENCE TO RELATED APPLICATION

This application claims priority to U.S. Provisional Patent Application No. 6/0, filed Mar. 31, 2023, the entire contents of which is incorporated herein by reference.

### TECHNICAL FIELD

This document generally relates to consumable components for cytology, and in particular, to a sample vial used for preparing the sample for processing.

### BACKGROUND

Cytology (also known as cytopathology) involves examining cells from bodily tissues or fluids to determine a diagnosis. A certain kind of physician, called a pathologist, will look at the cells in the tissue sample under a microscope and look for characteristics or abnormalities in the cells. Since cytology only examines cells, which are so tiny, pathologists only need a very small sample of tissue to do a cytology test.

Standard procedure in collecting, preparing and analyzing biofluid samples for cytodiagnostics includes biofluid specimens being collected from the patient and transported to a cytopathology laboratory. Freshly collected samples are prepared with multiple centrifugation and manual handling steps including cell fixing, washing, and cytochemical staining. These are necessary steps for preparing cell-based assays including cell smears, cell blocks and cell solutions. Prepared samples undergo microscopic examination, flow cytometry, and cytogenetic analysis.

### SUMMARY

Embodiments of the disclosed technology are directed to consumable components for cytology, which include a sample vial. In an example, the consumable sample vial is designed to receive a sample volume between 10 and 750 μL, receive a buffer solution, and enable mixing of the sample with the buffer solution prior to allowing a user to deposit some amount of the mixture into another consumable for subsequent processing, e.g., cell deposition and staining. The remaining mixture may be securely stored and transported using the sample vial. The sample vial is manufactured from a transparent and flexible material that allows the user to observe the fluid and mixture levels in the vial and squeeze to dispense the mixture.

In an example aspect, a sample vial for sample collection and deposition includes a vial body, a body opening, a nozzle, a labeling area comprising a second flat portion joined to the first flat portion and in a same plane as the first flat portion, and a gripping area comprising a third flat portion joined to the second flat portion and in a plane perpendicular to the second flat portion. Herein, (a) the vial body comprises a bottom portion, a top portion, and a sidewall extending from the top portion to the bottom portion, wherein the bottom portion and the sidewall forms a flexible chamber, and the body opening comprises a circular opening joined to the top portion of the vial body and a first flat portion extending outward from a circumference of the circular opening, and (b) the nozzle consists of a nozzle cap comprising a rim configured to fit into the circular opening, a nozzle shaft extending from the nozzle cap and terminating in a nozzle tip, and a nozzle tether connecting the nozzle cap to the first flat portion.

In another example aspect, a system for collecting and depositing a sample includes a sample vial and a sample vial rack that consists of a base comprising a lower ledge, a sample vial holding structure comprising (a) an upper ledge positioned above and parallel to the lower ledge and (b) a panel connecting a distal end of the upper ledge and a distal end of the lower ledge, wherein a proximal end of the upper ledge comprises a plurality of keyhole-shaped openings, each keyhole-shaped opening comprising a hole with a diameter greater than a diameter of the sample vial and a notch that is wider than a thickness of the gripping area, and one or more magnet holders on a rear surface of the panel.

In yet another example aspect, a method of collecting and depositing a sample that uses the described sample vial includes affixing a label onto the labeling area, loading a buffer solution, via the body opening, into the vial body, loading the sample into the vial body, homogenizing, using the vial body, the buffer solution and the sample to create a homogenized buffer solution and sample mix, and dispensing the homogenized buffer solution and sample mix.

The above and other aspects and their implementations are described in greater detail in the drawings, the descriptions, and the claims.

### BRIEF DESCRIPTION OF THE DRAWINGS

FIG. 1 shows an example embodiment of a sample vial.

FIG. 2 shows an example of a workflow that uses the sample vial.

FIGS. 3A-3G shows examples of holding the sample vial of FIG. 1 during different stages of the workflow illustrated in FIG. 2.

FIGS. 4A-4C show alternative embodiments of the sample vial.

FIGS. 5A-5C show different lid and cap configurations for embodiments of the sample vial.

FIGS. 6A-6C show different views of an example sample vial rack used to hold one or more sample vials.

FIGS. 7A-7C show alternative embodiments for the sample vial rack.

FIGS. 8A and 8B are diagrams that highlight certain design features of an example sample vial rack.

FIG. 9 shows a flowchart of an example method of collecting and depositing a sample into a sample vial.

### DETAILED DESCRIPTION

Biofluids are liquid-based cellular solutions that originate from the human body. Examples include urine, blood, pleural fluid, peritoneal fluid, cerebrospinal fluid, aspirates from tumors in the body, and wound exudates. Sample preparation and analysis of biofluids is emerging as an application area that many microfluidic technologies may be able to address. Microfluidic research has in particular focused on the preparation and analysis of blood. While significant diagnostic information about the patient can be obtained from blood analysis, other biofluids may be as rich in terms of diagnostic information. These other biofluids present unique sample preparation and analysis challenges that microfluidic technologies are poised to address.

Sample collection is the first step in preparing a sample for analysis, in which the collection technique can often impact downstream cytodiagnostic assays that can be performed. Samples are obtained through collection techniques that vary in their level of invasiveness and risk to the patient. Sample collection techniques include phlebotomy, fluid aspiration via needle or syringe, or saline washing of a mucosal surface with catheter assistance. Depending on the procedure that is required, sample collection is carried out in different locations, and can be performed in a patient procedure room, at the hospital bedside of the patient, at an outpatient office, or in the clinical laboratory.

Embodiments of the disclosed technology provide a sample vial that enables the efficient and consistent collection and initial processing of samples that can then be further prepared for cytological examination, as well as a sample vial rack, which serves as an accessory to hold multiple sample vials, allows for their easy removal, and ensures visibility of the bottom of the vials.

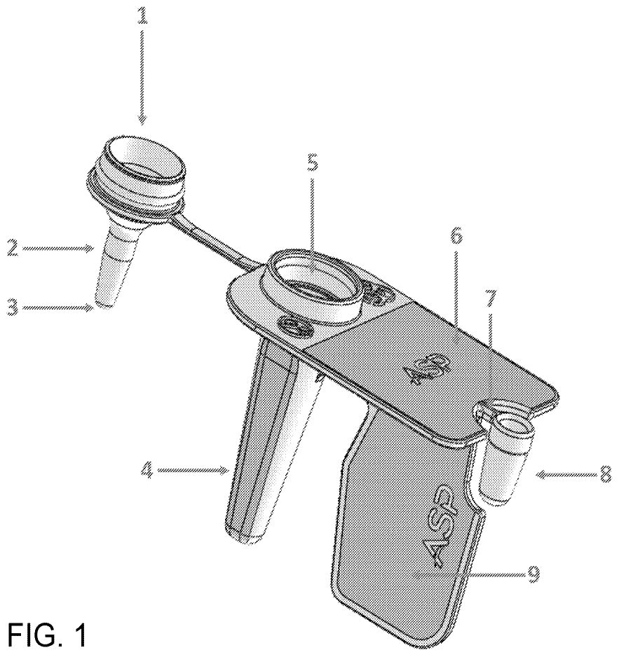

FIG. 1 shows an example embodiment of a sample vial, in accordance with the disclosed technology. As shown therein, the sample vial includes the following features and/or components:
nozzle cap (1)
nozzle shaft (2)
nozzle tip (3)
vial body (4)
body opening (5)
labeling area (6)
lid tether (7)
vial lid (8)
gripping area (9)

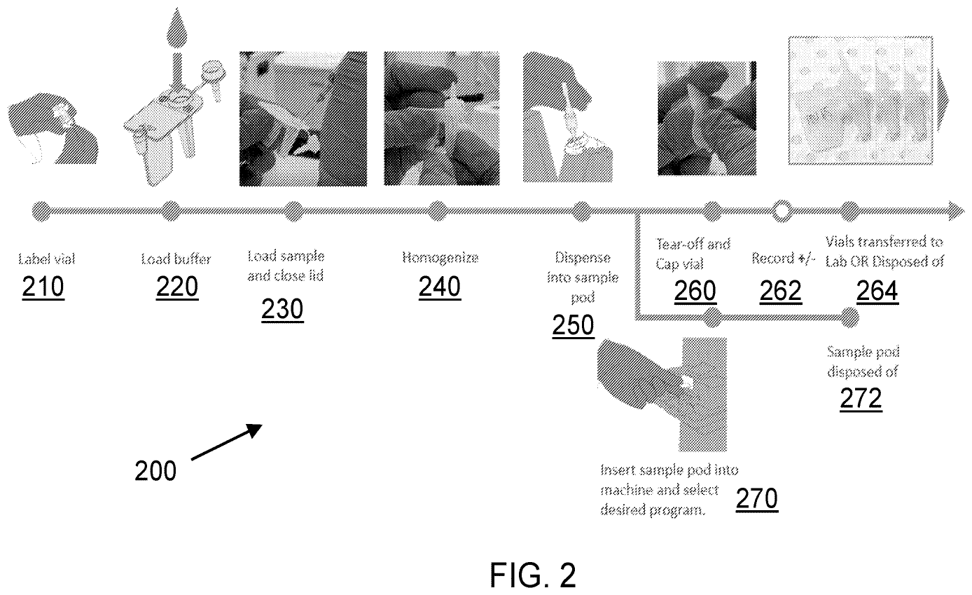

Herein, the rim of the nozzle cap (1) fits snugly into the vial body opening (5), and the nozzle cap (5) aids in the dispensing of the homogenized sample/buffer mix into the sample pod (described in the context of the sample vial workflow in FIG. 2).

The nozzle shaft (2) fits snugly with the vial lid (8), thereby allowing the vial to be sealed. The nozzle shaft (2) ends in a nozzle tip (3) that allows the homogenized sample/buffer mix to be squeezed out in small, controlled volumes.

The vial body (4) is a flexible chamber where the sample alone or the sample and the buffer can be mixed by massaging. This is also where the user (or operator) squeezes to dispense the homogenized sample/buffer mix via the nozzle shaft (2) and nozzle tip (3). The body opening (5) is the mouth of the vial body (4), and allows the sample and the buffer to be inserted for homogenization.

The labeling area (6) is an area-approximately the size of a slide labeling area-that can be written on or have a sticker affixed thereto. The labeling area (6) is physically connected to the vial lid (8) via a lid tether (7). The lid tether (7) is a small, integrated tear-off feature which holds the lid until the operator needs to seal the vial, and the vial lid (8) pushes over the nozzle shaft (2) to seal the sample vial, and keeping the sample contained for storage and subsequent analysis. In an example, the lid tether (7) is designed to break (or tear) off with a small number of rotations, e.g., 3-5 rotations can break the vial tether (7) and free the vial lid (8).

The labeling area (6) is perpendicular to a gripping area (9), which allows the user to hold on to the sample vial without disturbing the contents of vial body (4). In an example, the gripping area (9) and the labeling area are designed (e.g., with a concave edge instead of a straight edge) to add clearance around the vial lid (8) for easier user access. In another example, the gripping area (9) is designed with ridges to make gripping easier.

In some embodiments, the sample vial can be used to prepare and dispense a mix of the buffer solution and the sample. In other embodiments, the sample vial can be used to prepare and dispense only the sample, e.g., a liquid sample might not require a buffer solution to be added.

In some embodiments, the sample vial shown in FIG. 1 is an integrated, single consumable that is created from a material that is resilient even with very thin wall sections and having a low surface energy to ensure the effective removal of the sample after processing. This ensures that none of the collected sample goes to waste, e.g., after dispensing a portion of the homogenized buffer and sample mix into a sample pod, all the remainder of the sample can be transferred to a CytoLyt or formalin jar or commonly used sample transfer liquid. In an example, the material is thermoplastic polyurethane, polyvinyl chloride (plasticized), low-density polyethylene, or polypropylene.

In some embodiments, the dimensions of sample vial shown in FIG. 1 can be configured as follows:
the thickness at the bottom portion of the vial body (4) is about 0.3 mm, which ensures that the sample in the bottom portion of the vial can be easily massaged.
the thickness of the vial tether (7) is about 0.15 mm, which enables the vial cap (8) to be torn (or broken) off easily. In some scenarios, the tooling processes used to manufacture the sample vial can support a minimum thickness of 0.15 mm.
the thickness of the labeling area (6) and the gripping area (9) is about 1.2 mm to provide additional stiffness to those regions of the sample vial.

FIG. 2 shows an example of a workflow 200 that uses the sample vial. As shown therein, the workflow 200 includes, at operation 210, labeling the sample vial on the labeling area (6). Optionally, at least some portion of the information on the sample vial label is also included on the labels on the CytoLyt jar or formalin jar, and/or the sample pod (also referred to as the specimen input port (SIP)).

The workflow 200 includes, at operation 220, loading the sample vial body (4) with buffer. In an example, the sample vial body (4) is prefilled (or loaded) with 20 μL of buffer (e.g., phosphate-buffered saline (PBS)). At operation 230, the workflow 200 includes loading the sample into the vial body (4). In an example, the entire contents of the fine-needle aspiration (FNA) needle are deposited into the vial body (4).

The workflow 200 includes, at operation 240, homogenizing the buffer and the sample by, for example, massaging the vial body (4). The homogenized sample and buffer mix is then squeezed out in small, controlled volumes into the sample pod via the nozzle shaft (2) and nozzle tip (3). In an example, three drops of the homogenized sample and buffer mix can be squeezed out into the sample pod.

The workflow 200 can then include, at operations 260-264, tearing (or breaking) off the vial tether (7) to free the vial lid (8) to seal the vial, recording additional information, and either transferring the sample vial to a laboratory for further analysis or disposing of the sample vial.

Alternatively, the workflow 200 can then include, at operations 270 and 272, placing the sample pod into the machine and selecting the desired program for analysis, and then disposing of the sample pod. Any remaining homogenized sample and buffer mix in the sample vial can be transferred to a CytoLyt jar or formalin jar, which can be taken to the laboratory for further analysis.

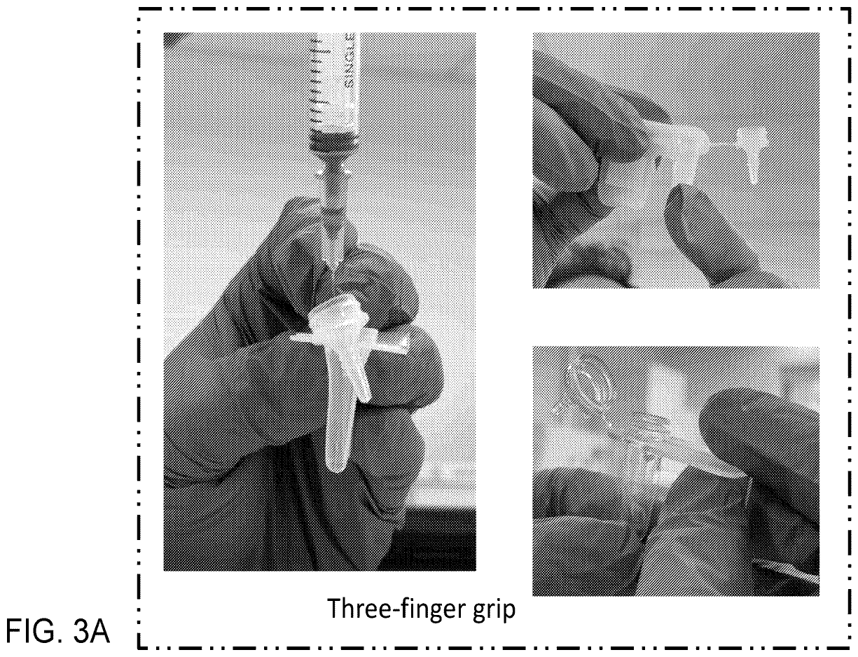

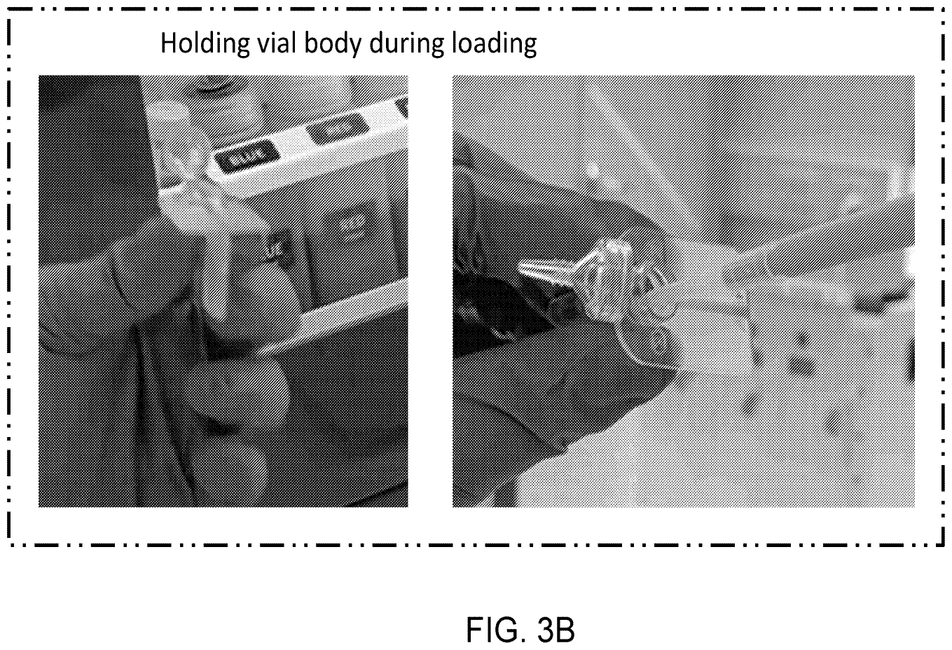

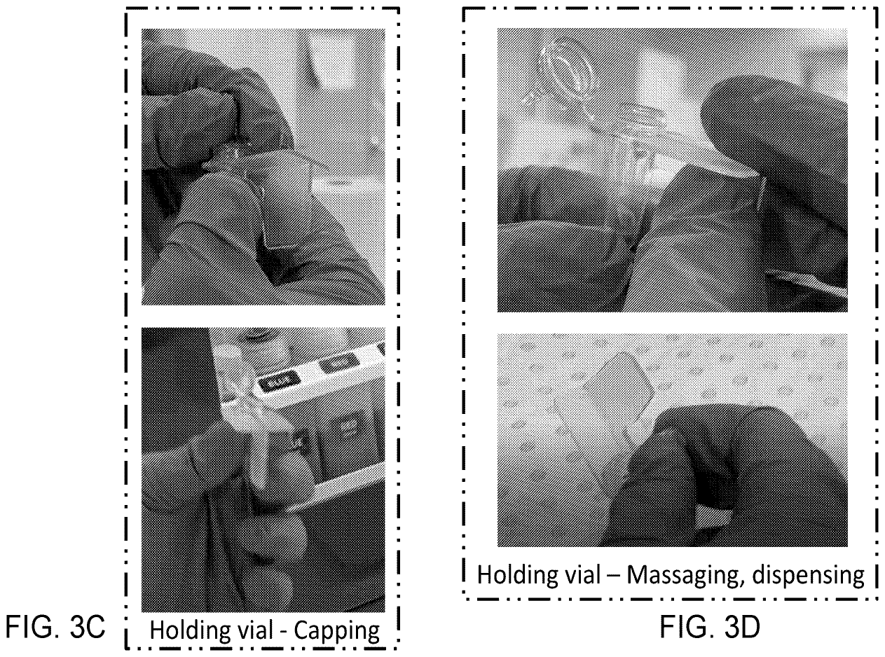

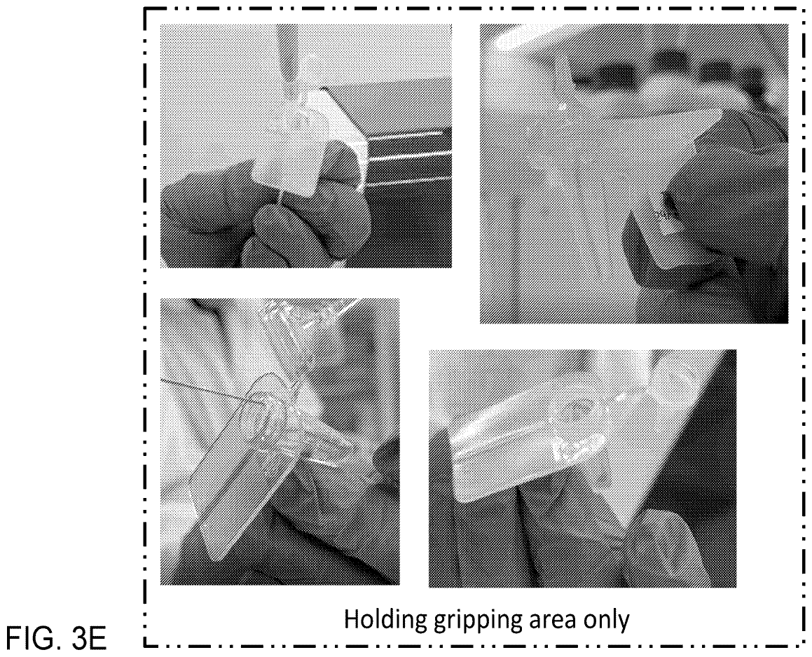

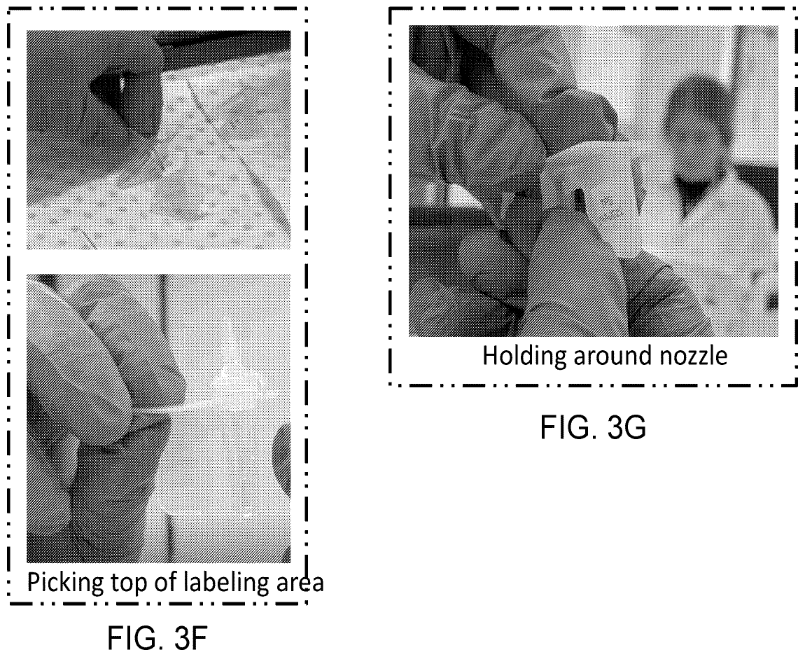

FIGS. 3A-3G shows examples of holding the sample vial of FIG. 1 during different stages of the workflow illustrated in FIG. 2. FIG. 3A shows a three-finger grip that can be used when loading the sample (e.g., from an FNA needle) into the sample body of the sample vial, and massaging the vial body to homogenize the buffer solution and sample mix. FIG. 3B shows a grip that is used to hold the sample vial when loading. FIGS. 3C and 3D show the grips that are used when capping and massaging (or dispensing) the homogenized buffer solution and sample mix, respectively. FIG. 3E shows the various functions that can be performed by holding only the gripping area of the sample vial. FIG. 3F shows that the sample vial can be manipulated by only holding onto the top portion of the labeling area, and FIG. 3G shows holding the sample vial around the nozzle haft.

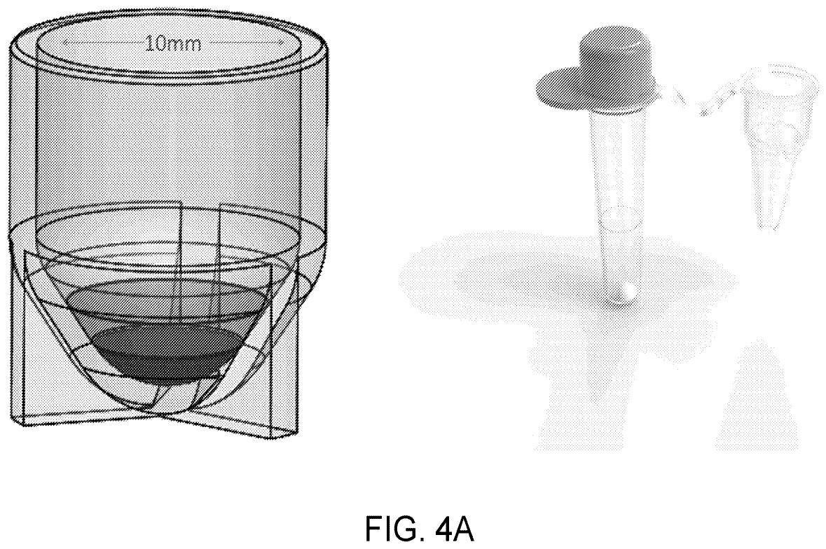

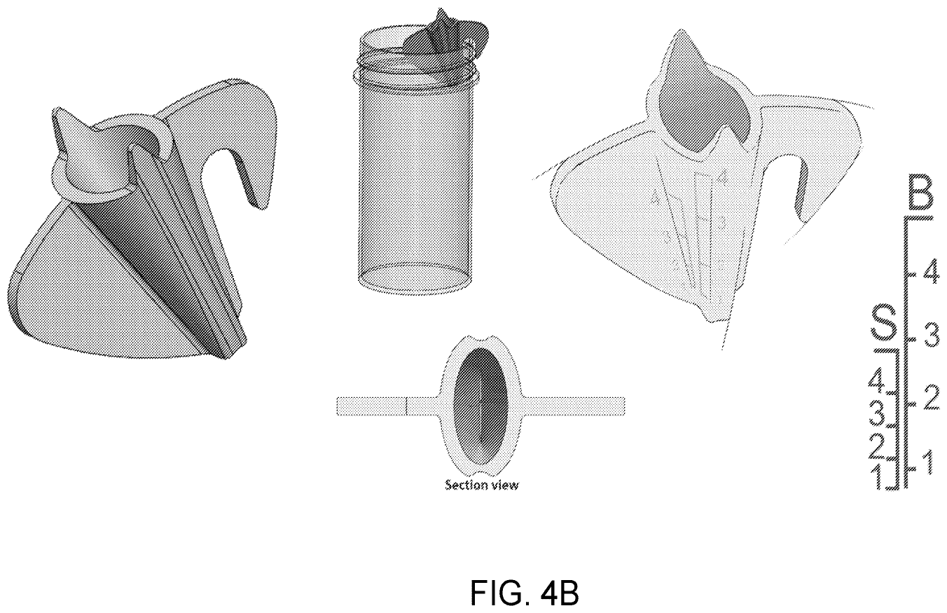

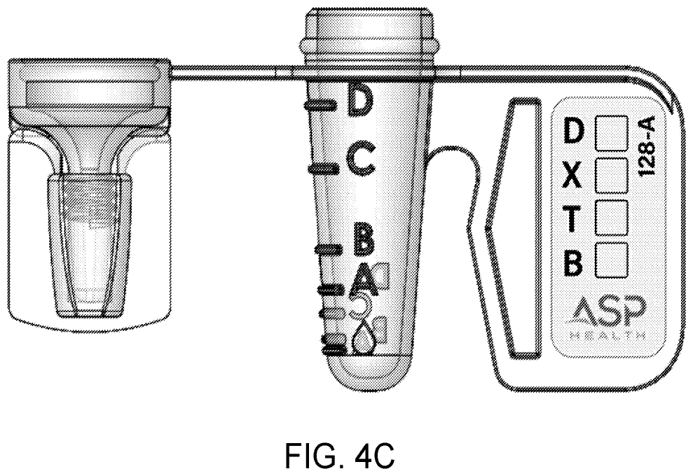

FIGS. 4A-4C show alternative embodiments of the sample vial.

FIG. 4A shows an example sample vial with greater than 500 μL capacity and volume gradations. As illustrated, the base is wide enough so that the sample vial is freestanding. In different use cases, it can rest on the bench, hang from a CytoLyt jar, or be capped and sent to the laboratory for processing. The taper on the lower portion of the jar is designed based on one or more of the following factors: viscosity of the buffer solution, viscosity of the collected sample, amount of sample being collected, etc.

FIG. 4B shows an example sample vial that has living hinges for easy mixing, no nozzle but a spout to control where and who quickly the homogenized buffer solution and sample mix can be dispensed. In some embodiments, the sample vial in FIG. 4B is made of a material (e.g., an elastomeric material) that allows the inside volume of sample vial to be collapsed completely to push out small samples (or remnants of the homogenized buffer solution and sample mix within the sample vial). In other embodiments, the sample vial in FIG. 4B can have volume graduations (or fill lines) that clearly indicate how much buffer solution should be added for a given volume of the collected sample.

FIG. 4C shows an example of a sample vial that is specifically designed for transportation to a remote laboratory, and includes (a) an easy twist top with wings such that when the lid is fully screwed on, its wings snap over two ribs on the nozzle, keeping the contents secure, and (b) a labeling area on the handle for identifying information. In an example, the labeling area measures about 10 mm×20 mm.

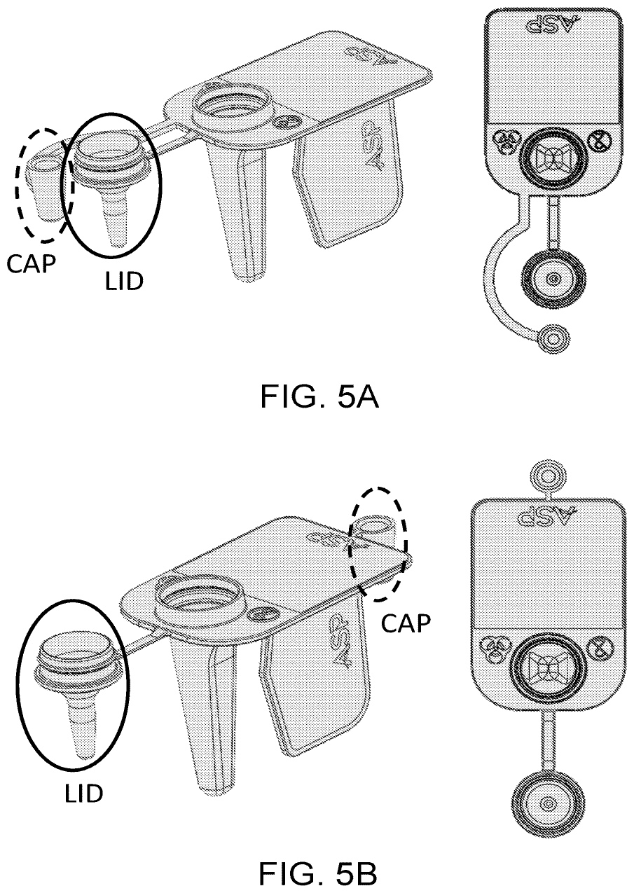

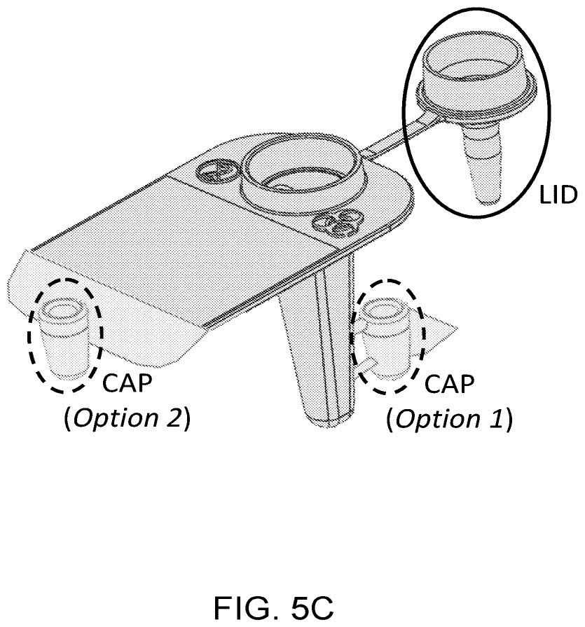

FIGS. 5A-5C show different nozzle lid (or cap) configurations (with respect to the nozzle shaft and nozzle tip, or collectively, the nozzle) for embodiments of the sample vial. As shown therein, the nozzle lid is tethered adjacent to the nozzle, with both the nozzle cap and nozzle being tethered from the same side of the labeling area (FIG. 5A), the nozzle lid is tethered diametrically opposite from the nozzle (FIG. 5B), and the nozzle lid is either tethered from the vial body (FIG. 5C, Option 1) or tethered diametrically opposite from the nozzle on a bendable portion of the labeling area (FIG. 5C, Option 2). Each of the configurations shown in FIGS. 5A-5C were designed to improve handling and usage by the operator (or user). For example, the tethering of the nozzle cap must make it easy for the user to break off the nozzle cap with an intuitive movement, the tether for the nozzle cap must be easy to manufacture (e.g., and results in the embodiment shown in FIG. 5A being less preferable for this reason), etc.

Embodiments of the sample vial, described in the context of FIGS. 1-5, need to routinely be placed (or set aside) on the bench for short durations of time as other procedures not involving the collected sample are carried out. To that end, the described embodiments include a sample vial rack that can hold multiple sample vials upright, allows for their easy removal from the sample vial rack, and ensures visibility of the bottom of each of the vial bodies.

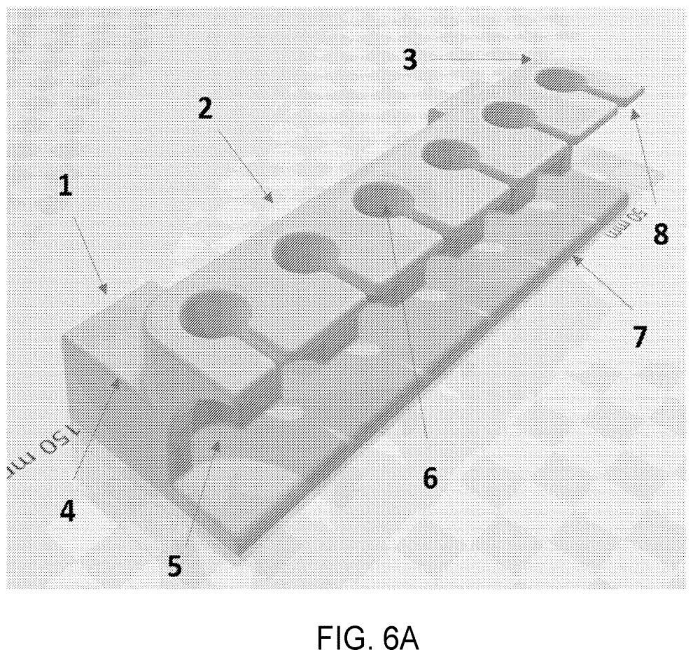

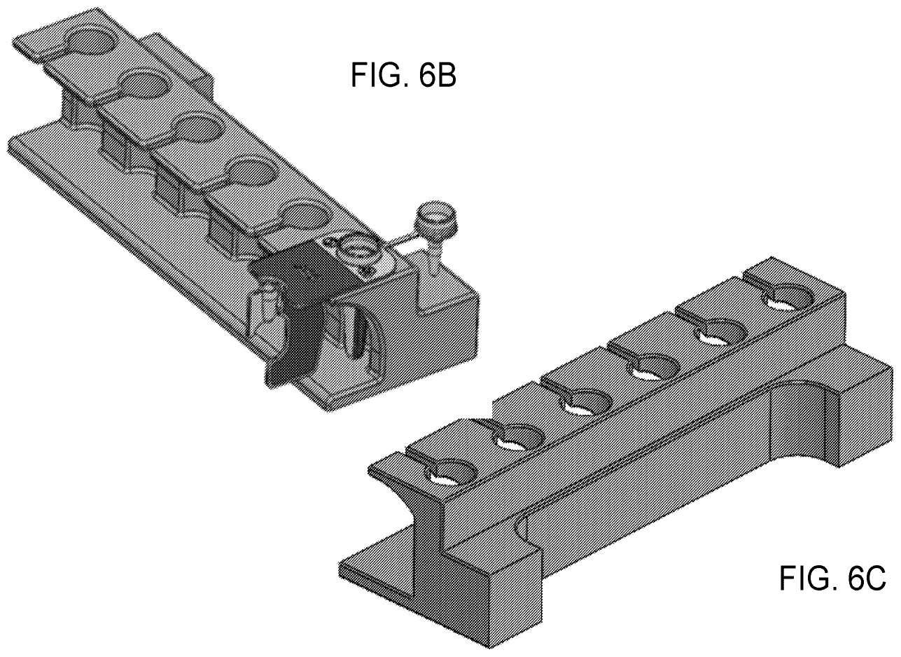

FIGS. 6A-6C show different views of an example of a sample vial rack used to hold the sample vials. As shown in FIGS. 6A-6C, the sample vial rack includes the following features and/or components:
magnet area (1)
empty space (2)
rounded edges and fillets (3)
recessed nozzle ledge (4)
vial visibility (5)
vial holes (6)
lower ledge (7)
upper ledge (8)

Herein, the two protruding magnet areas (1) have a circular holder where the magnets can be glued. This allows for magnetic mounting to the deposition and staining device without the nozzles getting in the way or the magnets getting pulled off the part. The empty spaces (2) help with easier cleaning and reduce manufacturing costs, and were created by removing unused external and internal parts of the sample vial rack. The sample vial rack features rounded edges and fillets (3) to improve the appearance, ergonomics, and usability.

The recessed back (or nozzle) ledge (4) allows the nozzle caps of the vial to rest comfortably without pressing against the side of the device where it is mounted or affecting the position of the vial body. The cutout areas of holes allows for more grip space when holding the vial in place, as well as provides a full-frontal view of the bottom of the vial (5) when loading the buffer solution and/or sample during the sample preparation process. The keyhole-shaped vial holes (6) allow for easy holding of the sample vials, and their easy removal.

The lower ledge (7) of the sample vial rack provides structural support and stability to the overall part, as well as a resting ledge for the bottom of the vial to sit on. A width of the upper ledge (8) has been optimized to allow for some support under the labeling face of the sample vial. At the same time, it allows enough grip space and finger clearance to easily remove the sample vials when lifting the sample vials vertically out of the sample vial rack.

In some embodiments, the sample vial rack is manufactured using a stereolithography (SLA) 3D printer, and uses a printer ink selected from Accura Xtreme Gray, Accura 25, and Accura SL 7820. In other embodiments, the sample vial rack is manufactured using an injection molding process, and uses a material that provides strength and a smooth finish, e.g., polyoxymethylene (POM) or acrylonitrile butadiene styrene (ABS).

In some embodiments, the thinnest portion of the sample vial rack corresponds to the back portion of the magnet area (1), which is kept at about 0.1 mm to ensure that the magnet that is being used to temporarily affix the sample vial rack to the metal side of a deposition and staining unit is most effective. The default thickness for the other portions of the sample vial rack is about 2.5 mm. More generally, the ratio of the thickness of the other regions to the magnet region of the rack will be kept at 10:4.

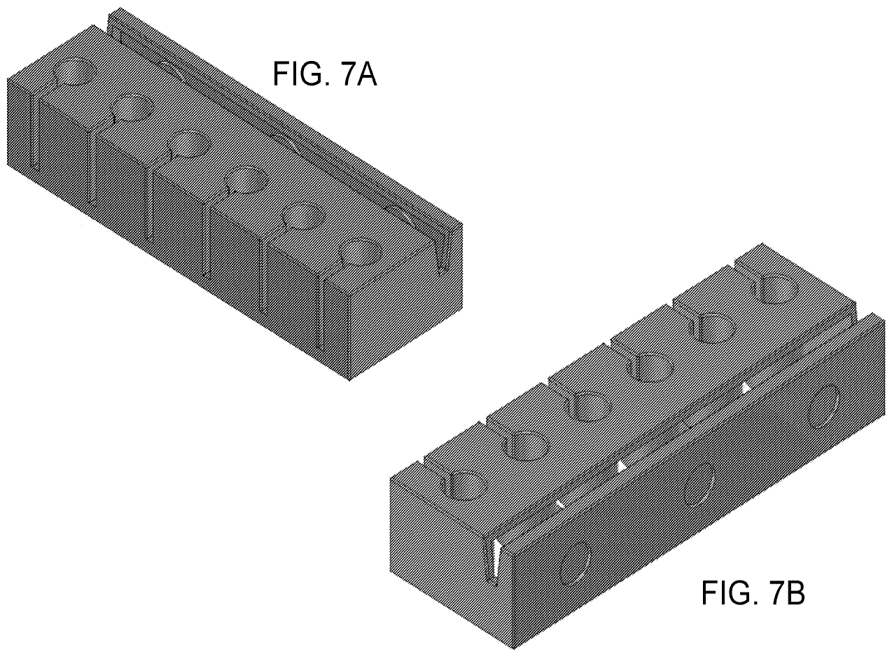

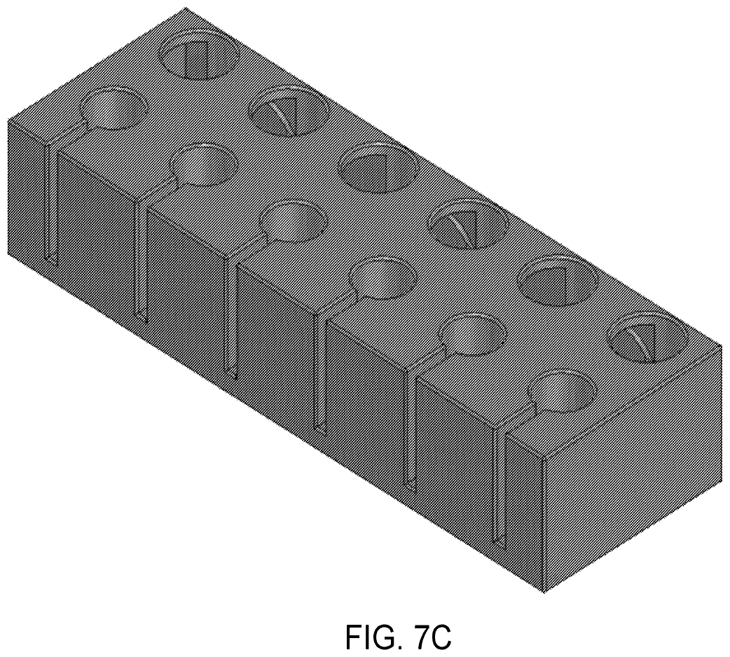

In some embodiments, the recessed nozzle ledge (4) of the embodiment illustrated in FIGS. 6A-6C can be substituted with a slot or individual holes. Using either the slot for the nozzles (FIGS. 7A and 7B) or the individual holes for the nozzles (FIG. 7C) enables the nozzles of the sample vials to be placed in the sample vial rack without them interfering with either the placement or the removal of the sample vials in the open (or uncapped) configuration.

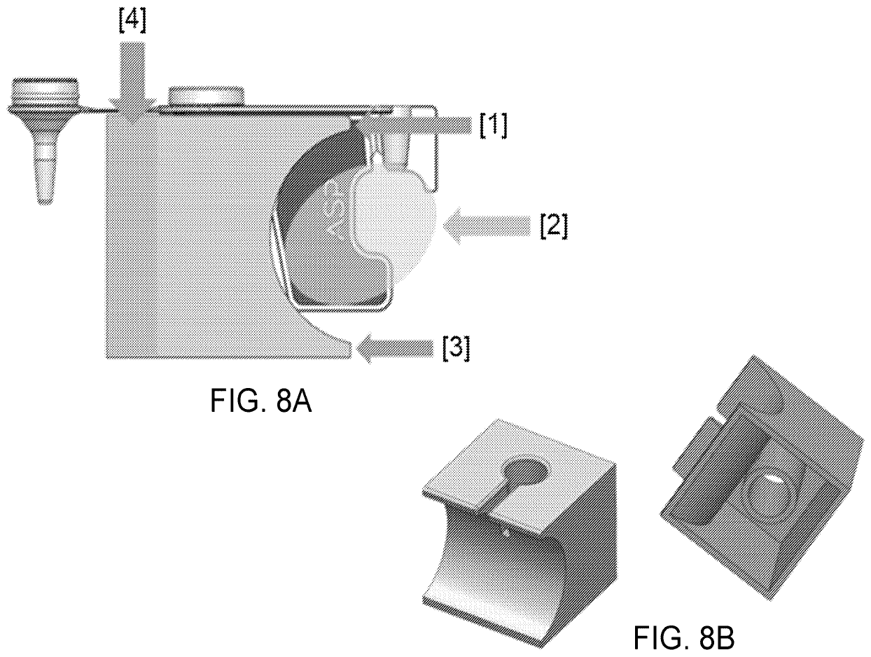

FIGS. 8A and 8B are diagrams that highlight certain design features of an example sample vial rack. As shown in FIG. 8A, the sample vial rack is designed to ensure that [1] there is support underneath the labeling area of the sample vial, [2] the grip area has plenty of empty space to allow the user (or operator) to easily grip the gripping area of the sample vial during placement or removal, [3] support at the foot of the sample vial rack for stability, and [4] an extended portion of the sample vial rack to mitigate the nozzle from flopping around. In the embodiments shown in FIGS. 6A-6C, the feature labeled [4] has been replaced with the recessed back (or nozzle) ledge (4).

FIG. 8B shows that the sample vial rack has been shelled from the bottom for material efficiency, cost, and print time.

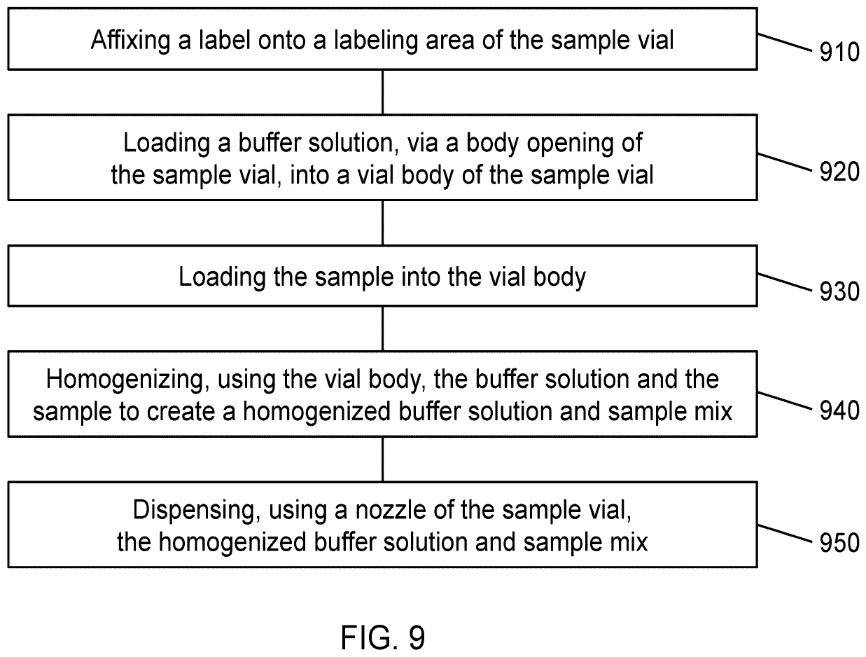

FIG. 9 is a flowchart of an example method 900 for collecting and depositing a sample into a sample vial. The method 900 includes, at operation 910, affixing a label onto a labeling area of the sample vial.

The method 900 includes, at operation 920, loading a buffer solution, via a body opening of the sample vial, into a vial body of the sample vial, and at operation 930, loading the sample into the vial body.

The method 900 includes, at operation 940, homogenizing, using the vial body, the buffer solution and the sample to create a homogenized buffer solution and sample mix.

The method 900 includes, at operation 950, dispensing, using a nozzle of the sample vial, the homogenized buffer solution and sample mix.

Embodiments of the disclosed technology provide, in some aspects, the following technical solutions:
P1. An apparatus for collecting and depositing a sample, comprising a sample vial comprising a vial body comprising a bottom portion, a top portion, and a sidewall extending from the top portion to the bottom portion, wherein the bottom portion and the sidewall forms a flexible chamber, and a body opening comprising a circular opening joined to the top portion of the vial body and a first flat portion extending outward from a circumference of the circular opening; a nozzle comprising a nozzle cap comprising a rim configured to fit into the circular opening, a nozzle shaft extending from the nozzle cap and terminating in a nozzle tip, and a nozzle tether connecting the nozzle cap to the first flat portion; a labeling area comprising a second flat portion joined to the first flat portion and in a same plane as the first flat portion; a gripping area comprising a third flat portion joined to the second flat portion and in a plane perpendicular to the second flat portion.
P2. The apparatus of solution P1, further comprising a vial lid configured to cover the nozzle tip and seal the nozzle shaft; and a lid tether connecting the vial lid to either the first flat portion or the second flat portion.
P3. The apparatus of solution P2, wherein a thickness of the lid tether is about 0.15 mm.
P4. The apparatus of any of solutions P1 to P3, wherein a thickness of the bottom portion is about 0.3 mm and a thickness of the labeling area and/or the gripping area is about 1.2 mm.
P5. The apparatus of any of the preceding solutions, wherein the nozzle tip is self-closing.
P6. The apparatus of any of the preceding solutions, wherein a size of the labeling area is substantially similar to a size of a label for a cytopathology slide.
P7. The apparatus of any of the preceding solutions, wherein the rim of the nozzle cap includes a first set of grooves configured to interlock with a second set of grooves on the circular opening of the sample vial.
P8. The apparatus of any of the preceding solutions, wherein a capacity of the flexible chamber is less than 800 μL.
P9. The apparatus of any of the preceding solutions, wherein the sample vial is made from thermoplastic polyurethane, plasticized polyvinyl chloride, low-density polyethylene, or polypropylene.
P10. A system for collecting and depositing a sample, comprising a sample vial as described in any of solutions P1 to P9; and a sample vial rack, comprising a base comprising a lower ledge, a sample vial holding structure comprising (a) an upper ledge positioned above and parallel to the lower ledge and (b) a panel connecting a distal end of the upper ledge and a distal end of the lower ledge, wherein a proximal end of the upper ledge comprises a plurality of keyhole-shaped openings, each keyhole-shaped opening comprising a hole with a diameter greater than a diameter of the sample vial and a notch that is wider than a thickness of the gripping area, and one or more magnet holders on a rear surface of the panel.
P11. The system of solution P10, wherein the one or more magnet holders are configured to removably attach the sample vial rack to a sample deposition and staining system.
P12. The system of any of the preceding solutions, wherein a thickness of the upper ledge and/or the lower ledge is about 2.5 mm and a thickness of each of the one or more magnet holders is about 1.0 mm.
P13. The system of any of the preceding solutions, wherein the one or more magnet holders comprise an upper surface configured to support the nozzle cap of the sample vial when the sample vial has been placed in one of the plurality of keyhole-shaped openings.
P14. The system of any of solutions P10 to P13, wherein the sample vial rack is made from a plastic printing material that is compatible with stereolithography three-dimensional (3D) printing.
P15. The system of any of solutions P10 to P13, wherein the sample vial rack is made from polyoxymethylene or acrylonitrile butadiene styrene (ABS).
P16. A method of using the sample vial in any of solutions P1 to P9 in conjunction with the sample vial rack in any of solutions P10 to P15 to prepare the sample for deposition and staining.
P17. A method of collecting and depositing a sample that uses the sample vial in any of solutions P1 to P9, the method comprising affixing a label onto the labeling area; loading a buffer solution, via the body opening, into the vial body; loading the sample into the vial body; homogenizing, using the vial body, the buffer solution and the sample to create a homogenized buffer solution and sample mix; and dispensing the homogenized buffer solution and sample mix.
P18. The method of solution P17, further comprising dispensing the homogenized buffer solution and sample mix into a sample pod.
P19. The method of solution P17, further comprising detaching, by breaking the nozzle tether, the nozzle cap; and sealing the sample vial by snugly fitting the nozzle cap to the body opening.
P20. The method of any of solutions P17 to P19, wherein the sample is collected using fine-needle aspiration (FNA).

Embodiments of the disclosed technology provide, in some aspects, the following additional technical solutions:
N1. An apparatus for collecting and depositing a sample, comprising a sample vial comprising a vial body comprising a bottom portion, a top portion, and a sidewall extending from the top portion to the bottom portion, wherein the bottom portion and the sidewall forms a flexible chamber, and a body opening comprising a circular opening joined to the top portion of the vial body and a first flat portion extending outward from a circumference of the circular opening; a nozzle comprising a nozzle cap comprising a rim configured to fit into the circular opening, a nozzle shaft extending from the nozzle cap and terminating in a nozzle tip, and a nozzle tether connecting the nozzle cap to the first flat portion; a labeling area comprising a second flat portion joined to the first flat portion and in a same plane as the first flat portion; and a gripping area comprising a third flat portion joined to the second flat portion and in a plane perpendicular to the second flat portion.
N2. An apparatus for collecting and depositing a sample, comprising a vial body comprising a bottom portion, a top portion, and a sidewall extending from the top portion to the bottom portion, wherein the bottom portion and the sidewall forms a flexible chamber; a body opening comprising a circular opening joined to the top portion of the vial body and a first flat portion extending outward from a circumference of the circular opening; a nozzle comprising a nozzle cap comprising a rim configured to fit into the circular opening, a nozzle shaft extending from the nozzle cap and terminating in a nozzle tip, and a nozzle tether connecting the nozzle cap to the first flat portion; a labeling area comprising a second flat portion joined to the first flat portion and in a same plane as the first flat portion; and a gripping area comprising a third flat portion joined to the second flat portion and in a plane perpendicular to the second flat portion.
N3. The apparatus of solution N1 or N2, further comprising a vial lid configured to cover the nozzle tip and seal the nozzle shaft; and a lid tether connecting the vial lid to either the first flat portion or the second flat portion.
N4. The apparatus of solution N3, wherein a thickness of the lid tether is about 0.15 mm.
N5. The apparatus of any of solutions N1 to N4, wherein a thickness of the bottom portion is about 0.3 mm and a thickness of the labeling area and/or the gripping area is about 1.2 mm.
N6. The apparatus of any of solutions N1 to N5, wherein the nozzle tip is self-closing.
N7. The apparatus of any of solutions N1 to N6, wherein a size of the labeling area is substantially similar to a size of a label for a cytopathology slide.
N8. The apparatus of any of solutions N1 to N7, wherein the rim of the nozzle cap includes a first set of grooves configured to interlock with a second set of grooves on the circular opening of the sample vial.
N9. The apparatus of any of solutions N1 to N8, wherein a capacity of the flexible chamber is less than 800 μL.
N10. The apparatus of any of solutions N1 to N9, wherein the sample vial is made from thermoplastic polyurethane, plasticized polyvinyl chloride, low-density polyethylene, or polypropylene.
N11. A system for collecting a sample for deposition and staining, comprising a sample vial; and a sample vial rack, comprising a base comprising a lower ledge, a sample vial holding structure comprising (a) an upper ledge positioned above and parallel to the lower ledge and (b) a panel connecting a distal end of the upper ledge and a distal end of the lower ledge, wherein a proximal end of the upper ledge comprises a plurality of keyhole-shaped openings, each keyhole-shaped opening comprising a hole with a diameter greater than a diameter of the sample vial and a notch that is wider than a thickness of a gripping area of the sample vial, and one or more magnet holders on a rear surface of the panel.
N12. A system for collecting a sample for deposition and staining, comprising a sample vial comprising a vial body comprising a bottom portion, a top portion, and a sidewall extending from the top portion to the bottom portion, wherein the bottom portion and the sidewall forms a flexible chamber, a body opening comprising a circular opening joined to the top portion of the vial body and a first flat portion extending outward from a circumference of the circular opening, a nozzle comprising a nozzle cap comprising a rim configured to fit into the circular opening, a nozzle shaft extending from the nozzle cap and terminating in a nozzle tip, and a nozzle tether connecting the nozzle cap to the first flat portion, a labeling area comprising a second flat portion joined to the first flat portion and in a same plane as the first flat portion, and a gripping area comprising a third flat portion joined to the second flat portion and in a plane perpendicular to the second flat portion; and a sample vial rack, comprising a base comprising a lower ledge, a sample vial holding structure comprising (a) an upper ledge positioned above and parallel to the lower ledge and (b) a panel connecting a distal end of the upper ledge and a distal end of the lower ledge, wherein a proximal end of the upper ledge comprises a plurality of keyhole-shaped openings, each keyhole-shaped opening comprising a hole with a diameter greater than a diameter of the sample vial and a notch that is wider than a thickness of the gripping area, and one or more magnet holders on a rear surface of the panel.
N13. The system of solution N11 or N12, wherein the one or more magnet holders are configured to removably attach the sample vial rack to a sample deposition and staining system.
N14. The system of any of solutions N11 to N13, wherein a thickness of the upper ledge and/or the lower ledge is about 2.5 mm and a thickness of each of the one or more magnet holders is about 1.0 mm.
N15. The system of any of solutions N11 to N14, wherein the one or more magnet holders comprise an upper surface configured to support the nozzle cap of the sample vial when the sample vial has been placed in one of the plurality of keyhole-shaped openings.
N16. The system of any of solutions N11 to N15, wherein the sample vial rack is made from a plastic printing material that is compatible with stereolithography three-dimensional (3D) printing.
N17. The system of any of solutions N11 to N15, wherein the sample vial rack is made from polyoxymethylene or acrylonitrile butadiene styrene (ABS).
N18. A method of collecting and depositing a sample into a sample vial, wherein the sample vial comprises a vial body comprising a bottom portion, a top portion, and a sidewall extending from the top portion to the bottom portion, wherein the bottom portion and the sidewall forms a flexible chamber, a body opening comprising a circular opening joined to the top portion of the vial body and a first flat portion extending outward from a circumference of the circular opening, a nozzle comprising a nozzle cap comprising a rim configured to fit into the circular opening, a nozzle shaft extending from the nozzle cap and terminating in a nozzle tip, and a nozzle tether connecting the nozzle cap to the first flat portion, a labeling area comprising a second flat portion joined to the first flat portion and in a same plane as the first flat portion, and a gripping area comprising a third flat portion joined to the second flat portion and in a plane perpendicular to the second flat portion, and wherein the method comprises affixing a label onto the labeling area; loading a buffer solution, via the body opening, into the vial body; loading the sample into the vial body; homogenizing, using the vial body, the buffer solution and the sample to create a homogenized buffer solution and sample mix; and dispensing the homogenized buffer solution and sample mix.
N19. A method of collecting and depositing a sample into a sample vial, comprising affixing a label onto a labeling area of the sample vial; loading a buffer solution, via a body opening of the sample vial, into a vial body of the sample vial; loading the sample into the vial body; homogenizing, using the vial body, the buffer solution and the sample to create a homogenized buffer solution and sample mix; and dispensing, using a nozzle of the sample vial, the homogenized buffer solution and sample mix.
N20. The method of solution N18 or N19, further comprising dispensing the homogenized buffer solution and sample mix into a sample pod.
N21. The method of solution N18 or N19, further comprising detaching, by breaking the nozzle tether, the nozzle cap; and sealing the sample vial by snugly fitting the nozzle cap to the body opening.
N22. The method of any of solutions N18 to N21, wherein the sample is collected using fine-needle aspiration (FNA).

Embodiments of the disclosed technology provide, in some aspects, the following additional technical solutions:
C1. A sample vial for collecting and depositing a sample, comprising: a vial body comprising a bottom portion, a top portion, and a sidewall extending from the top portion to the bottom portion, wherein the bottom portion and the sidewall forms a flexible chamber; a body opening comprising a circular opening joined to the top portion of the vial body and a first flat portion extending outward from a circumference of the circular opening; a nozzle comprising: a nozzle cap comprising a rim configured to fit into the circular opening, a nozzle shaft extending from the nozzle cap and terminating in a nozzle tip, and a nozzle tether connecting the nozzle cap to the first flat portion; a labeling area comprising a second flat portion joined to the first flat portion and in a same plane as the first flat portion; and a gripping area comprising a third flat portion joined to the second flat portion and in a plane perpendicular to the second flat portion.
C2. The sample vial of solution C1, further comprising: a vial lid configured to cover the nozzle tip and seal the nozzle shaft; and a lid tether connecting the vial lid to either the first flat portion or the second flat portion.
C3. The sample vial of solution C2, wherein a thickness of the lid tether is about 0.15 mm.
C4. The sample vial of any of solutions C1 to C3, wherein a thickness of the bottom portion is about 0.3 mm and a thickness of the labeling area and/or the gripping area is about 1.2 mm.
C5. The sample vial of any of solutions C1 to C3, wherein the nozzle tip is self-closing.
C6. The sample vial of any of solutions C1 to C3, wherein a size of the labeling area is substantially similar to a size of a label for a cytopathology slide.
C7. The sample vial of any of solutions C1 to C3, wherein the rim of the nozzle cap includes a first set of grooves configured to interlock with a second set of grooves on the circular opening of the sample vial.
C8. The sample vial of any of solutions C1 to C3, wherein a capacity of the flexible chamber is less than 800 μL.
C9. The sample vial of any of solutions C1 to C3, wherein the sample vial is made from thermoplastic polyurethane, plasticized polyvinyl chloride, low-density polyethylene, or polypropylene.
C10. A system for collecting a sample for deposition and staining, comprising: a sample vial comprising: a vial body comprising a bottom portion, a top portion, and a sidewall extending from the top portion to the bottom portion, wherein the bottom portion and the sidewall forms a flexible chamber, a body opening comprising a circular opening joined to the top portion of the vial body and a first flat portion extending outward from a circumference of the circular opening, a nozzle comprising: a nozzle cap comprising a rim configured to fit into the circular opening, a nozzle shaft extending from the nozzle cap and terminating in a nozzle tip, and a nozzle tether connecting the nozzle cap to the first flat portion, a labeling area comprising a second flat portion joined to the first flat portion and in a same plane as the first flat portion, and a gripping area comprising a third flat portion joined to the second flat portion and in a plane perpendicular to the second flat portion; and a sample vial rack, comprising: a base comprising a lower ledge, a sample vial holding structure comprising (a) an upper ledge positioned above and parallel to the lower ledge and (b) a panel connecting a distal end of the upper ledge and a distal end of the lower ledge, wherein a proximal end of the upper ledge comprises a plurality of keyhole-shaped openings, each keyhole-shaped opening comprising a hole with a diameter greater than a diameter of the sample vial and a notch that is wider than a thickness of the gripping area, and one or more magnet holders on a rear surface of the panel.
C11. The system of solution C10, wherein the one or more magnet holders are configured to removably attach the sample vial rack to a sample deposition and staining system.
C12. The system of solution C10, wherein a thickness of the upper ledge and/or the lower ledge is about 2.5 mm and a thickness of each of the one or more magnet holders is about 1.0 mm.
C13. The system of solution C10, wherein the one or more magnet holders comprise an upper surface configured to support the nozzle cap of the sample vial when the sample vial has been placed in one of the plurality of keyhole-shaped openings.
C14. The system of any of solutions C10 to C13, wherein the sample vial rack is made from a plastic printing material that is compatible with stereolithography three-dimensional (3D) printing.
C15. The system of any of solutions C10 to C13, wherein the sample vial rack is made from polyoxymethylene or acrylonitrile butadiene styrene (ABS).
C16. A method of collecting and depositing a sample into a sample vial, wherein the sample vial comprises: a vial body comprising a bottom portion, a top portion, and a sidewall extending from the top portion to the bottom portion, wherein the bottom portion and the sidewall forms a flexible chamber, a body opening comprising a circular opening joined to the top portion of the vial body and a first flat portion extending outward from a circumference of the circular opening, a nozzle comprising: a nozzle cap comprising a rim configured to fit into the circular opening, a nozzle shaft extending from the nozzle cap and terminating in a nozzle tip, and a nozzle tether connecting the nozzle cap to the first flat portion, a labeling area comprising a second flat portion joined to the first flat portion and in a same plane as the first flat portion, and a gripping area comprising a third flat portion joined to the second flat portion and in a plane perpendicular to the second flat portion, and wherein the method comprises: affixing a label onto the labeling area; loading a buffer solution, via the body opening, into the vial body; loading the sample into the vial body; homogenizing, using the vial body, the buffer solution and the sample to create a homogenized buffer solution and sample mix; and dispensing the homogenized buffer solution and sample mix.
C17. The method of solution C16, further comprising: dispensing the homogenized buffer solution and sample mix into a sample pod.
C18. The method of solution C16, further comprising: detaching, by breaking the nozzle tether, the nozzle cap; and sealing the sample vial by snugly fitting the nozzle cap to the body opening.
C19. The method of any of solutions C16 to C18, wherein the sample is collected using fine-needle aspiration (FNA).
C20. An apparatus for sample collection and deposition that implements the method of any one or more of solutions C16 to C19.

In this document, the term “cellular sample” refers to any biological sample containing cells. Cellular samples can be a tissue sample or samples (e.g., any collection of cells) removed from a subject. The tissue sample can be a collection of interconnected cells that perform a similar function within an organism. A cellular sample can also be any solid or fluid sample obtained from, excreted by, or secreted by any living organism, including, without limitation, single-celled organisms, such as bacteria, yeast, protozoans, and amebae, multicellular organisms (such as plants or animals, including samples from a healthy or apparently healthy human subject or a human patient affected by a condition or disease to be diagnosed or investigated, such as cancer). In some embodiments, a cellular sample is mountable on a microscope slide and includes, without limitation, a section of tissue, an organ, a tumor section, a smear, a frozen section, a cytology prep, or cell lines. An incisional biopsy, a core biopsy, an excisional biopsy, a needle aspiration biopsy (e.g., fine needle aspiration (FNA)), a core needle biopsy, a stereotactic biopsy, an open biopsy, or a surgical biopsy can be used to obtain the sample.

The detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A sample vial for collecting and depositing a sample, comprising:
a vial body comprising a bottom portion, a top portion, and a sidewall extending from the top portion to the bottom portion, wherein the bottom portion and the sidewall forms a flexible chamber;
a body opening comprising a circular opening joined to the top portion of the vial body and a first flat portion extending outward from a circumference of the circular opening;
a nozzle comprising:
a nozzle cap comprising a rim configured to fit into the circular opening,
a nozzle shaft extending from the nozzle cap and terminating in a nozzle tip, and
a nozzle tether connecting the nozzle cap to the first flat portion;
a labeling area comprising a second flat portion joined to the first flat portion and in a same plane as the first flat portion; and
a gripping area comprising a third flat portion joined to the second flat portion and in a plane perpendicular to the second flat portion.

2. The sample vial of claim 1, further comprising:
a vial lid configured to cover the nozzle tip and seal the nozzle shaft; and
a lid tether connecting the vial lid to either the first flat portion or the second flat portion.

3. The sample vial of claim 2, wherein a thickness of the lid tether is about 0.15 mm.

4. The sample vial of claim 1, wherein a thickness of the bottom portion is about 0.3 mm and a thickness of the labeling area and/or the gripping area is about 1.2 mm.

5. The sample vial of claim 1, wherein the nozzle tip is self-closing.

6. The sample vial of claim 1, wherein a size of the labeling area is substantially similar to a size of a label for a cytopathology slide.

7. The sample vial of claim 1, wherein the rim of the nozzle cap includes a first set of grooves configured to interlock with a second set of grooves on the circular opening of the sample vial.

8. The sample vial of claim 1, wherein a capacity of the flexible chamber is less than 800 μL.

9. The sample vial of claim 1, wherein the sample vial is made from thermoplastic polyurethane, plasticized polyvinyl chloride, low-density polyethylene, or polypropylene.

10. A system for collecting a sample for deposition and staining, comprising:
a sample vial comprising:
a vial body comprising a bottom portion, a top portion, and a sidewall extending from the top portion to the bottom portion, wherein the bottom portion and the sidewall forms a flexible chamber,
a body opening comprising a circular opening joined to the top portion of the vial body and a first flat portion extending outward from a circumference of the circular opening,
a nozzle comprising:
a nozzle cap comprising a rim configured to fit into the circular opening,
a nozzle shaft extending from the nozzle cap and terminating in a nozzle tip, and
a nozzle tether connecting the nozzle cap to the first flat portion,
a labeling area comprising a second flat portion joined to the first flat portion and in a same plane as the first flat portion, and
a gripping area comprising a third flat portion joined to the second flat portion and in a plane perpendicular to the second flat portion; and
a sample vial rack, comprising:
a base comprising a lower ledge,
a sample vial holding structure comprising (a) an upper ledge positioned above and parallel to the lower ledge and (b) a panel connecting a distal end of the upper ledge and a distal end of the lower ledge, wherein a proximal end of the upper ledge comprises a plurality of keyhole-shaped openings, each keyhole-shaped opening comprising a hole with a diameter greater than a diameter of the sample vial and a notch that is wider than a thickness of the gripping area, and
one or more magnet holders on a rear surface of the panel.

11. The system of claim 10, wherein the one or more magnet holders are configured to removably attach the sample vial rack to a sample deposition and staining system.

12. The system of claim 10, wherein a thickness of the upper ledge and/or the lower ledge is about 2.5 mm and a thickness of each of the one or more magnet holders is about 1.0 mm.

13. The system of claim 10, wherein the one or more magnet holders comprise an upper surface configured to support the nozzle cap of the sample vial when the sample vial has been placed in one of the plurality of keyhole-shaped openings.

14. The system of claim 10, wherein the sample vial rack is made from a plastic printing material that is compatible with stereolithography three-dimensional (3D) printing.

15. The system of claim 10, wherein the sample vial rack is made from polyoxymethylene or acrylonitrile butadiene styrene (ABS).

16. A method of collecting and depositing a sample into a sample vial,
wherein the sample vial comprises:
a vial body comprising a bottom portion, a top portion, and a sidewall extending from the top portion to the bottom portion, wherein the bottom portion and the sidewall forms a flexible chamber,
a body opening comprising a circular opening joined to the top portion of the vial body and a first flat portion extending outward from a circumference of the circular opening,
a nozzle comprising:
a nozzle cap comprising a rim configured to fit into the circular opening,
a nozzle shaft extending from the nozzle cap and terminating in a nozzle tip, and
a nozzle tether connecting the nozzle cap to the first flat portion,
a labeling area comprising a second flat portion joined to the first flat portion and in a same plane as the first flat portion, and
a gripping area comprising a third flat portion joined to the second flat portion and in a plane perpendicular to the second flat portion, and
wherein the method comprises:
affixing a label onto the labeling area;
loading a buffer solution, via the body opening, into the vial body;
loading the sample into the vial body;
homogenizing, using the vial body, the buffer solution and the sample to create a homogenized buffer solution and sample mix; and
dispensing the homogenized buffer solution and sample mix.

17. The method of claim 16, further comprising:
dispensing the homogenized buffer solution and sample mix into a sample pod.

18. The method of claim 16, further comprising:
detaching, by breaking the nozzle tether, the nozzle cap; and
sealing the sample vial by snugly fitting the nozzle cap to the body opening.

19. The method of claim 16, wherein the sample is collected using fine-needle aspiration (FNA).

20. (canceled)
